(19) United States
(12) Patent Application Publication
(10) Pub. No.: US 2009/0326409 A1
(43) Pub. Date: Dec. 31, 2009

(54) Clothing sufficiency indicator

(76) Inventors: Jason C. Cohen, Appleton, WI (US); Corey Thomas Cunningham, Larsen, WI (US); David W. Koenig, Menasha, WI (US); Thomas Michael Ales, III, Neenah, WI (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/215,445

(22) Filed: Jun. 27, 2008

(51) Int. Cl. A61B 5/01; A61F 13/15

(52) U.S. Cl. 600/549; 604/385.01

(57) ABSTRACT

A clothing sufficiency indication system is presented for use in conjunction with a disposable absorbent article. The system includes a microclimate sensor adapted to be disposed on the disposable absorbent article, the microclimate sensor including a microclimate temperature sensing portion adapted to sense a microclimate temperature, wherein the clothing sufficiency indication system is adapted to generate a clothing sufficiency indication based on the microclimate temperature. Also presented is a method for determining the clothing sufficiency of a subject including providing a clothing sufficiency indication system including a microclimate sensor for measuring a microclimate temperature within clothing of the subject, translating the microclimate temperature into a clothing sufficiency recommendation, and communicating the clothing sufficiency recommendation to a caregiver.

### BACKGROUND

This disclosure relates to an apparatus and method for reassuring a caregiver that a baby or other non-communicating subject is sufficiently but not overly clothed or otherwise covered. The apparatus and method use objective measurements indicative of the temperature of the microclimate between the subject and the subject's clothing.

Caregivers of babies, particularly newborns, and other non-communicative subjects have a strong need to understand whether the subjects are sufficiently clothed. First-time parents in particular are not always sure how much clothing to put on their child. This has become more of a concern with the new guidelines regarding no blankets, sheets, etc. allowed in cribs.

Prior devices have attempted to measure skin temperature, but this is of little use to a caregiver who simply wants to know if more or less clothing would be advantageous.

While particular aspects and/or individual features of the present disclosure have been illustrated and described, it would be obvious to those skilled in the art that various other changes and modifications can be made without departing from the spirit and scope of the disclosure. Further, it should be apparent that all combinations of such aspects and features are possible and can result in preferred executions of the disclosure.

### SUMMARY

Caregivers can be provided with reassurance and a sense of security by providing safe, comfortable, easy ways for a caregiver to care for a subject. Giving a caregiver a tool to help evaluate whether a subject is sufficiently clothed will help reassure the caregiver with respect to how well a subject is tolerating an environmental temperature and provide the caregiver with a sense of security regarding the subject's health. The present disclosure solves the problems outlined above by providing an apparatus and method for reassuring a caregiver that a subject is sufficiently clothed or covered. The apparatus and method use objective measurements indicative of the temperature of the microclimate between the subject's body and the subject's clothing.

In one aspect of the present disclosure, a clothing sufficiency indication system for use in conjunction with a disposable absorbent article for a subject having skin includes a microclimate sensor adapted to be disposed on the disposable absorbent article, the microclimate sensor including a microclimate temperature sensing portion adapted to sense a microclimate temperature, wherein the clothing sufficiency indication system is adapted to generate a clothing sufficiency indication based on the microclimate temperature.

In another aspect of the present disclosure, the disclosure includes a method for determining the clothing sufficiency of a subject including providing a clothing sufficiency indication system including a microclimate sensor for measuring a microclimate temperature within clothing of the subject, translating the microclimate temperature into a clothing sufficiency recommendation, and communicating the clothing sufficiency recommendation to a caregiver.

In another aspect of the present disclosure, the disclosure includes a method for determining the source of an elevated microclimate temperature of a microclimate within clothing, the method including measuring the microclimate temperature, measuring a skin temperature of skin within the clothing, determining a heat flux between the skin and the microclimate, and informing a caregiver whether the elevated microclimate temperature is caused by an external source or a body source.

In another aspect of the present disclosure, the disclosure includes a method for determining the source of an elevated microclimate temperature of a microclimate within clothing, the method including measuring the microclimate temperature, measuring an ambient temperature outside the clothing, determining a heat flux between the microclimate and an environment outside the clothing, and informing a caregiver whether the elevated microclimate temperature is caused by an external source or a body source.

### BRIEF DESCRIPTION OF THE DRAWINGS

The foregoing and other features and aspects of the present disclosure and the manner of attaining them will become more apparent, and the disclosure itself will be better understood by reference to the following description, appended claims and accompanying drawings, where:

FIG. 1 shows a rear perspective view of one aspect of an absorbent article and an aspect of the clothing sufficiency indication system of the present disclosure.

FIG. 2 shows a perspective, schematic view of an aspect of the clothing sufficiency indication system of the present disclosure.

FIG. 3 shows a perspective view of a sensor of the clothing sufficiency indication system illustrated in FIG. 2.

FIG. 4 shows a schematic view of an indication scheme of the clothing sufficiency indication system illustrated in FIGS. 1 and 2.

FIG. 5 shows a schematic view of an indication scheme of the clothing sufficiency indication system illustrated in FIGS. 1 and 2.

Repeat use of reference characters in the present specification and drawings is intended to represent the same or analogous features or elements of the present disclosure. The drawings are representational and are not necessarily drawn to scale. Certain proportions thereof may be exaggerated, while others may be minimized.

### DETAILED DESCRIPTION

It is to be understood by one of ordinary skill in the art that the present discussion is a description of exemplary aspects of the present disclosure only, and is not intended as limiting the broader aspects of the present disclosure.

For the purposes of this disclosure, “caregiver” is defined as the person who is taking care of a subject, whether that person is a parent, a healthcare worker, or other similar person. Similarly, the term “subject” refers to a subject of any age who cannot communicate temperature feeling in a meaningful manner.

For the purposes of this disclosure, “clothing” refers not only to garments worn by a subject but also to a diaper or other absorbent article and to any blanket, covering, outerwear, hat, mittens, foot coverings, or other articles used to keep a subject warm.

For the purposes of this disclosure, “ambient environment” refers to the environment or space outside the clothing in question, such as the room or other space in which the subject is found.

This disclosure describes a clothing sufficiency indication system to be used in conjunction with a diaper or other clothing for the purpose of monitoring a subject's environment to ensure that the subject has the proper amount of clothing or other coverings. This indication system produces, for example, a low reading if the subject has insufficient coverings by determining that the subject's microclimate (the environment adjacent the subject's body and within clothing) is too cold. Similarly, the indication system produces, for example, a high reading if the subject has excessive coverings that could overheat the subject by determining that the subject's microclimate is too hot. Likewise, the indication system produces, for example, an acceptable reading if the subject has appropriate coverings by determining that the subject's microclimate is within a proper temperature range. This determination may be based solely on the temperature of the subject's microclimate, or in reference to separately measured core/skin and/or ambient temperatures.

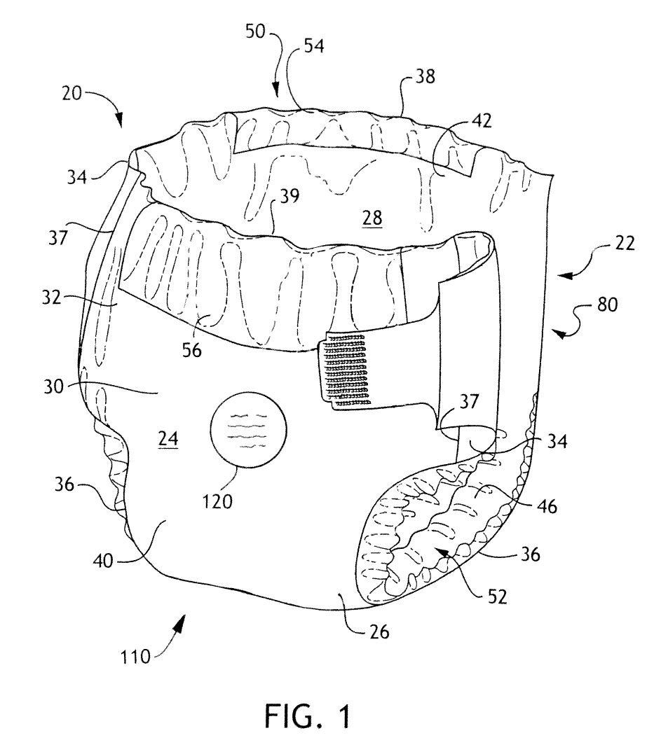

Referring to FIG. 1, for exemplary purposes, an absorbent article 20 that may be made in accordance with the present disclosure is shown. The absorbent article description provided herein is for illustrative purposes and should not in any way be used to limit or define the scope of the present disclosure. The absorbent article 20 may or may not be disposable. It is understood that the present disclosure is suitable for use with various other absorbent articles intended for personal wear including, but not limited to, diapers, training pants, swim pants, feminine hygiene products, incontinence products, medical garments, surgical pads and bandages, other personal care or health care garments, and the like, without departing from the scope of the present disclosure.

A diaper 20 is representatively illustrated in FIG. 1 in a partially fastened condition. The diaper 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The diaper 20 also defines an inner surface 28 adapted in use (e.g., positioned relative to the other components of the article 20) to be disposed toward the wearer, and an outer surface 30 opposite the inner surface. The front and back regions 22, 24 are those portions of the diaper 20 which, when worn, wholly or partially cover or encircle the waist or mid-lower torso of the wearer. The crotch region 26 generally is that portion of the diaper 20 which, when worn, is positioned between the legs of the wearer and covers the lower torso and crotch of the wearer. The absorbent article 20 has a pair of laterally opposite side edges 36 and a pair of longitudinally opposite waist edges, respectively designated front waist edge 38 and back waist edge 39.

The illustrated diaper 20 includes a chassis 32 that, in this aspect, encompasses the front region 22, the back region 24, and the crotch region 26. The chassis 32 includes an outer cover 40 and a bodyside liner 42 that may be joined to the outer cover 40 in a superimposed relation therewith by adhesives, ultrasonic bonds, thermal bonds or other conventional techniques. The chassis 32 may further include an absorbent structure (not shown) disposed between the outer cover 40 and the bodyside liner 42 for absorbing liquid body exudates exuded by the wearer, and may further include a pair of containment flaps 46 secured to the bodyside liner 42 for inhibiting the lateral flow of body exudates.

The elasticized containment flaps 46 define a partially unattached edge which assumes an upright configuration in at least the crotch region 26 of the diaper 20 to form a seal against the wearer's body. The containment flaps 46 can extend longitudinally along the entire length of the chassis 32 or may extend only partially along the length of the chassis.

To further enhance containment and/or absorption of body exudates, the diaper 20 may also suitably include leg elastic members (not shown). In some aspects, the absorbent article 20 may further include a surge management layer (not shown) that may be optionally located adjacent the absorbent structure 44 and attached to various components in the article 20, such as the absorbent structure 44 or the bodyside liner 42, by methods known in the art, such as by using an adhesive.

As shown in FIG. 1, the absorbent article 20 further includes a pair of opposing elastic side panels 34 that are attached to the back region of the chassis 32. The side panels 34 may be stretched around the waist and/or hips of a wearer to secure the garment in place. The elastic side panels are attached to the chassis along a pair of opposing longitudinal edges 37. The side panels 34 may be attached or bonded to the chassis 32 using any suitable bonding technique. For instance, the side panels 34 may be joined to the chassis by adhesives, ultrasonic bonds, thermal bonds, or other conventional techniques.

In an alternative aspect, the elastic side panels may also be integrally formed with the chassis 32. For instance, the side panels 34 may comprise an extension of the bodyside liner 42, of the outer cover 40, or of both the bodyside liner 42 and the outer cover 40.

In the aspects shown in the figures, the side panels 34 are connected to the back region of the absorbent article 20 and extend over the front region of the article when securing the article in place on a user. It should be understood, however, that the side panels 34 may alternatively be connected to the front region of the article 20 and extend over the back region when the article is donned.

With the absorbent article 20 in the fastened position as partially illustrated in FIG. 1, the elastic side panels 34 may be connected by a fastening system 80 to define a 3-dimensional diaper configuration having a waist opening 50 and a pair of leg openings 52. The waist opening 50 of the article 20 is defined by the waist edges 38 and 39 which encircle the waist of the wearer.

The side panels are releasably attachable to the front region 22 of the article 20 by the fastening system. It should be understood, however, that in other aspects, the side panels may be permanently joined to the chassis 32 at each end. The side panels may be permanently bonded together, for instance, when forming a training pant or absorbent swimwear.

In addition to possibly having elastic side panels, the absorbent article 20 may include various waist elastic members for providing elasticity around the waist opening. For example, as shown in the figures, the absorbent article 20 can include a front waist elastic member 54 and/or a back waist elastic member 56.

The materials used to form the absorbent article 20 that surround the waist elastic members 54 and 56 may vary depending upon the particular application and the particular product being produced.

FIG. 1 of the present disclosure illustrates a clothing sufficiency indication system 110 including a microclimate sensor 120 adapted to be disposed on the absorbent article 20. The microclimate sensor 120 includes a microclimate temperature sensing portion adapted to sense the temperature of the subject's microclimate, the environment adjacent the subject's body, typically the environment within any clothing the subject is wearing or covered with.

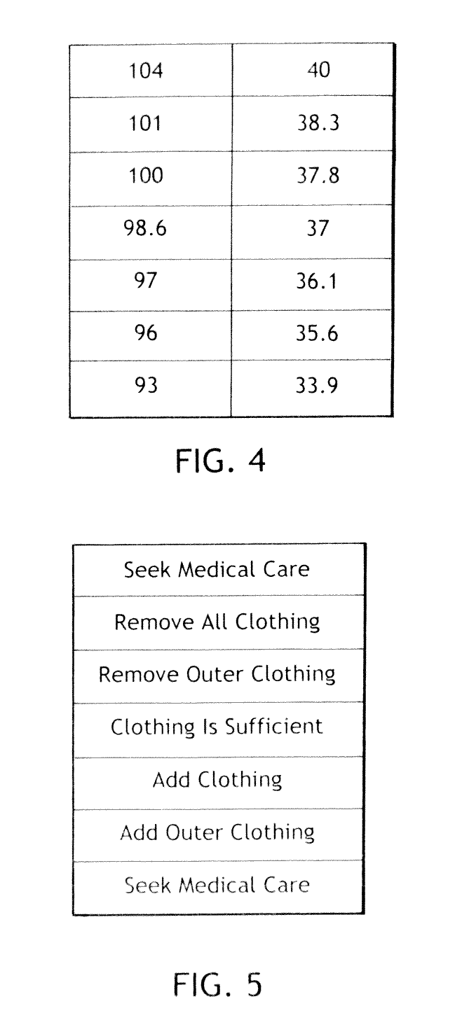

For the aspect in which the microclimate temperature is directly readable by the caregiver, the microclimate temperature can be displayed as illustrated, for example, in FIG. 4, with a temperature scale from hotter to colder in the downward direction. The values listed in FIG. 4 are merely for demonstration purposes and do not necessarily reflect the actual values chosen to be displayed for a commercial product. The temperature display may be in degrees Fahrenheit, degrees Celsius, both, or any other suitable temperature scale. In general, only one of the temperatures will be highlighted or visible so a caregiver will know which temperature applies.

In another aspect of the present disclosure, a single microclimate sensor 120 is used to monitor the microclimate between the subject and the subject's clothing. This microclimate sensor 120 serves as an indicator of the microclimate between the subject's skin and clothing. This microclimate sensor 120 can also be used to translate the microclimate temperature into an indication to the caregiver that the subject is too hot or too cold, thus indicating the need to remove or add clothing as appropriate to improve the microclimate.

For the aspect in which the microclimate temperature is translated into an indication of clothing sufficiency, which is directly readable by the caregiver, the clothing sufficiency can be displayed as illustrated, for example, in FIG. 5, with a translated temperature scale from hotter to colder in the downward direction. The descriptions listed in FIG. 5 are merely for demonstration purposes and do not necessarily reflect the actual descriptions chosen to be displayed for a commercial product. In general, only one of the descriptions will be highlighted or visible so a caregiver will know which description applies.

In various aspects of the present disclosure, the indication to the caregiver can be any suitable visual indicator including numeric figures (see FIG. 4), color scales, written descriptions (see FIG. 5), or character or other graphic representations such as red thermometers or blue ice cubes. The indication typically includes an indication of normal temperature or sufficient clothing. The indication can be any combination of these features or other suitable features. The clothing sufficiency indication system 110 can also include instructions to allow a caregiver to interpret the indication if the indication is not self-evident (e.g., the indication is a simple color scale or display).

The microclimate sensor 120 can be printed on or otherwise made a part of clothing, including an absorbent article 20. In addition, the microclimate sensor 120 can be any suitable electrical, chemical, or other sensor.

For example, a chemical-based microclimate sensor can incorporate both temperature sensitive and temperature insensitive materials. In one example, a printed scale using temperature insensitive inks is overlaid by a temperature sensitive coating such as a thermochromic dye. As the sensor warms, the thermochromic dye begins to melt, which causes it to become clear, revealing a printed scale underneath.

In another example, an electrical-based microclimate sensor can employ technologies known in the art for measuring temperature and displaying the measured temperature. Such a display can give the actual microclimate temperature or the display can indicate a temperature range. Additionally, an electrical-based microclimate sensor can be used to log or track the microclimate temperature the subject is experiencing over time.

In one aspect of the present invention, the microclimate sensor 120 translates the measured microclimate temperature into a recommendation ensuring that the subject has the proper amount of clothing or other coverings, such as that illustrated in FIG. 5. In one aspect, this recommendation may be made by simply replacing a temperature indication with a clothing recommendation. For example, if the microclimate temperature indication would otherwise indicate that the microclimate temperature is two degrees below normal temperature, the actual numerical microclimate temperature indication can be replaced by a short statement instructing the caregiver to add clothing. Additional indication schemes will be described below.

In another aspect of the present disclosure, the microclimate sensor 120 can be disposable to eliminate the need for keeping track of a durable sensor when not in use. The microclimate sensor 120 can be manufactured as a part of clothing or a disposable absorbent or other article.

In an exemplary and typical use, a caregiver places an absorbent article 20 on a subject, and then dresses the subject in typical clothing. When it comes time to change the absorbent article 20 of the subject, or at any time prior to that, the caregiver can remove all or part of the typical clothing and observe the microclimate sensor disposed on the outer surface of the absorbent article 20. The microclimate sensor 120 will indicate whether the subject has had sufficient clothing for the temperature conditions, and whether more or less clothing may be required.

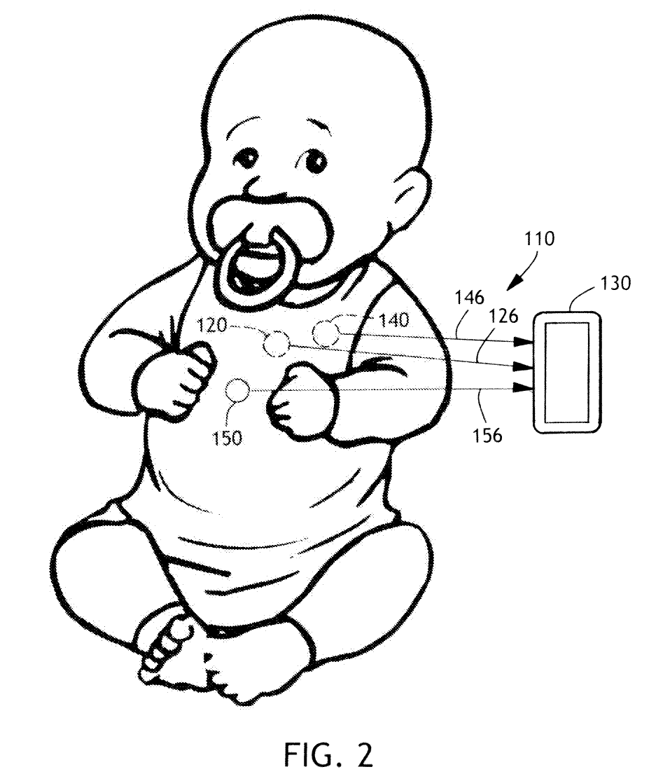

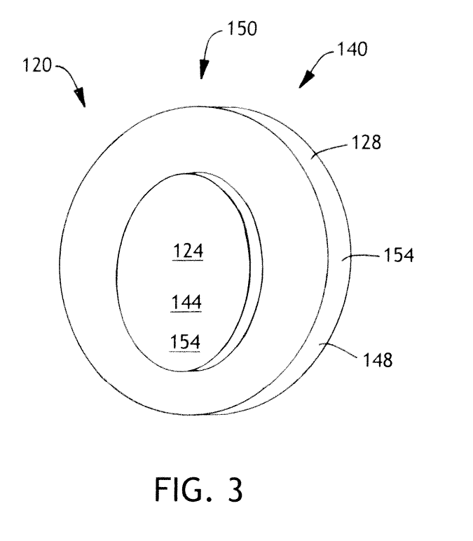

FIG. 2 of the present disclosure illustrates another aspect of the clothing sufficiency indication system 110 including a microclimate sensor 120 adapted to be disposed on or within clothing. The microclimate sensor 120 includes a microclimate temperature sensing portion 124 (see FIG. 3) adapted to sense the temperature of the subject's microclimate, the environment adjacent the subject's body. The microclimate sensor 120 then generates a microclimate temperature signal 126. In various aspects of the present disclosure, the microclimate temperature signal 126 can be directly readable by a caregiver as a microclimate temperature, or the microclimate temperature signal 126 can be transmitted to another device to be described below.

For the aspect in which the microclimate temperature is directly readable by the caregiver, the microclimate temperature can be displayed as illustrated, for example, in FIG. 4, with a temperature scale from hotter to colder in the downward direction. The values listed in FIG. 4 are merely for demonstration purposes and do not necessarily reflect the actual values chosen to be displayed for a commercial product. The temperature display may be in degrees Fahrenheit, degrees Celsius, both, or any other suitable temperature scale. In general, only one of the temperatures will be highlighted or visible so a caregiver will know which temperature applies.

As illustrated in FIG. 3, the microclimate sensor 120 can also include an insulator 128 to separate the microclimate temperature sensing portion 124 from the subject's skin so that the subject's skin temperature does not affect the temperature reading of the microclimate sensor 120. The microclimate sensor can also include a transmitter (not shown) for transmitting the microclimate temperature signal 126.

Returning to FIG. 2, and in one aspect of the present disclosure, a single microclimate sensor 120 is used to monitor the microclimate between the subject and the subject's clothing. This microclimate sensor 120 serves as an indicator of the microclimate between the subject's skin and clothing. This microclimate sensor 120 can also be used to translate the microclimate temperature into an indication to the caregiver that the subject is too hot or too cold, thus indicating the need to remove or add clothing as appropriate to improve the microclimate.

For the aspect in which the microclimate temperature signal 126 is translated into an indication of clothing sufficiency, which is directly readable by the caregiver, the clothing sufficiency can be displayed as illustrated, for example, in FIG. 5, with a translated temperature scale from hotter to colder in the downward direction. The descriptions listed in FIG. 5 are merely for demonstration purposes and do not necessarily reflect the actual descriptions chosen to be displayed for a commercial product. In general, only one of the descriptions will be highlighted or visible so a caregiver will know which description applies.

In various aspects of the present disclosure, the indication to the caregiver can be any suitable visual indicator including numeric figures (see FIG. 4), color scales, written descriptions (see FIG. 5), or character or other graphic representations such as red thermometers or blue ice cubes. The indication typically includes an indication of normal temperature or sufficient clothing. The indication can be any combination of these features or other suitable features.

The microclimate sensor 120 can be printed on or otherwise made a part of clothing, including an absorbent article. In addition, the microclimate sensor 120 can be any suitable electrical, chemical, or other sensor.

For example, a chemical-based microclimate sensor can incorporate both temperature sensitive and temperature insensitive materials. In one example, a printed scale using temperature insensitive inks is overlaid by a temperature sensitive coating such as a thermochromic dye. As the sensor warms, the thermochromic dye begins to melt, which causes it to become clear, revealing a printed scale underneath.

Any thermochromic substance that undergoes a color change at the desired temperature may generally be employed in the present disclosure. For example, liquid crystals may be employed as a thermochromic substance in some aspects. The wavelength of light (“color”) reflected by liquid crystals depends in part on the pitch of the helical structure of the liquid crystal molecules. Because the length of this pitch varies with temperature, the color of the liquid crystals is also a function of temperature. One particular type of liquid crystal that may be used in the present disclosure is a liquid crystal cholesterol derivative. Exemplary liquid crystal cholesterol derivatives may include alkanoic and aralkanoic acid esters of cholesterol, alkyl esters of cholesterol carbonate, cholesterol chloride, cholesterol bromide, cholesterol acetate, cholesterol oleate, cholesterol caprylate, cholesterol oleyl-carbonate, and so forth. Other suitable liquid crystal cholesterol derivatives are described in U.S. Pat. No. 3,600,060 to Churchill, et al.; U.S. Pat. No. 3,619,254 to Davis; and U.S. Pat. No. 4,022,706 to Davis, which are incorporated herein in their entirety by reference thereto for all purposes.

In addition to liquid crystals, another suitable thermochromic substance that may be employed in the present disclosure is a composition that includes a proton accepting chromogen (“Lewis base”) and a solvent. The melting point of the solvent controls the temperature at which the chromogen will change color. More specifically, at a temperature below the melting point of the solvent, the chromogen generally possesses a first color (e.g., red). When the solvent is heated to its melting temperature, the chromogen may become protonated or deprotonated, thereby resulting in a shift of the absorption maxima. The nature of the color change depends on a variety of factors, including the type of proton-accepting chromogen utilized and the presence of any additional temperature-insensitive chromogens. Regardless, the color change is typically reversible.

Although not required, the proton-accepting chromogen is typically an organic dye, such as a leuco dye. In solution, the protonated form of the leuco dye predominates at acidic pH levels (e.g., pH of about 4 or less). When the solution is made more alkaline through deprotonation, however, a color change occurs. Of course, the position of this equilibrium may be shifted with temperature when other components are present. Suitable leuco dyes for use in the present disclosure may include, for instance, phthalides; phthalanes; substituted phthalides or phthalanes, such as triphenylmethane phthalides, triphenylmethanes, or diphenylmethanes; acyl-leucomethylene blue compounds; fluoranes; indolylphthalides, spiropyranes; cumarins; and so forth. Exemplary fluoranes include, for instance, 3,3′-dimethoxyfluorane, 3,6-dimethoxyfluorane, 3,6-di-butoxyfluorane, 3-chloro-6-phenylamino-flourane, 3-diethylamino-6-dimethylfluorane, 3-diethylamino-6-methyl-7-chlorofluorane, and 3-diethyl-7,8-benzofluorane, 3,3′-bis-(p-dimethyl-aminophenyl)-7-phenylaminofluorane, 3-diethylamino-6-methyl-7-phenylamino-fluorane, 3-diethylamino-7-phenyl-aminofluorane, and 2-anilino-3-methyl-6-diethylamino-fluorane. Likewise, exemplary phthalides include 3,3′,3″-tris(p-dimethylamino-phenyl)phthalide, 3,3′-bis(p-dimethyl-aminophenyl)phthalide, 3,3-bis (p-diethylamino-phenyl)-6-dimethylamino-phthalide, 3-(4-diethylaminophenyl)-3-(1-ethyl-2-methylindol-3-yl)phthalide, and 3-(4-diethylamino-2-methyl)phenyl-3-(1,2-dimethylindol-3-yl)phthalide.

Although any solvent for the thermochromic dye may generally be employed in the present disclosure, it is typically desired that the solvent have a low volatility. For example, the solvent may have a boiling point of about 150° C. or higher, and in some aspects, from about 170° C. to 280° C. Likewise, the melting temperature of the solvent is also typically from about 25° C. to about 40° C., and in some aspects, from about 30° C. to about 37° C. Examples of suitable solvents may include saturated or unsaturated alcohols including about 6 to 30 carbon atoms, such as octyl alcohol, dodecyl alcohol, lauryl alcohol, cetyl alcohol, myristyl alcohol, stearyl alcohol, behenyl alcohol, geraniol, etc.; esters of saturated or unsaturated alcohols including about 6 to 30 carbon atoms, such as butyl stearate, methyl stearate, lauryl laurate, lauryl stearate, stearyl laurate, methyl myristate, decyl myristate, lauryl myristate, butyl stearate, lauryl palmitate, decyl palmitate, palmitic acid glyceride, etc.; azomethines, such as benzylideneaniline, benzylidenelaurylamide, o-methoxybenzylidene laurylamine, benzylidene p-toluidine, p-cumylbenzylidene, etc.; amides, such as acetamide, stearamide, etc.; and so forth.

The thermochromic composition may also include a proton-donating agent (also referred to as a “color developer”) to facilitate the reversibility of the color change. Such proton-donating agents may include, for instance, phenols, azoles, organic acids, esters of organic acids, and salts of organic acids. Exemplary phenols may include phenylphenol, bisphenol A, cresol, resorcinol, chlorolucinol, b-naphthol, 1,5-dihydroxynaphthalene, pyrocatechol, pyrogallol, trimer of p-chlorophenol-formaldehyde condensate, etc. Exemplary azoles may include benzotriaoles, such as 5-chlorobenzotriazole, 4-laurylaminosulfobenzotriazole, 5-butylbenzotriazole, dibenzotriazole, 2-oxybenzotriazole, 5-ethoxycarbonylbenzotriazole, etc.; imidazoles, such as oxybenzimidazole, etc.; tetrazoles; and so forth. Exemplary organic acids may include aromatic carboxylic acids, such as salicylic acid, methylenebissalicylic acid, resorcylic acid, gallic acid, benzoic acid, p-oxybenzoic acid, pyromellitic acid, b-naphthoic acid, tannic acid, toluic acid, trimellitic acid, phthalic acid, terephthalic acid, anthranilic acid, etc.; aliphatic carboxylic acids, such as stearic acid, 1,2-hydroxystearic acid, tartaric acid, citric acid, oxalic acid, lauric acid, etc.; and so forth. Exemplary esters may include alkyl esters of aromatic carboxylic acids in which the alkyl moiety has 1 to 6 carbon atoms, such as butyl gallate, ethyl p-hydroxybenzoate, methyl salicylate, etc.

The amount of the proton-accepting chromogen employed may generally vary, but is typically from about 2 wt. % to about 20 wt. %, and in some aspects, from about 5 to about 15 wt. % of the thermochromic substance. Likewise, the proton-donating agent may constitute from about 5 to about 40 wt. %, and in some aspects, from about 10 wt. % to about 30 wt. % of the thermochromic substance. In addition, the solvent may constitute from about 50 wt. % to about 95 wt. %, and in some aspects, from about 65 wt. % to about 85 wt. % of the thermochromic composition.

Regardless of the particular thermochromic substance employed, it may be microencapsulated to enhance the stability of the substance during processing. For example, the thermochromic substance may be mixed with a thermosetting resin according to any conventional method, such as interfacial polymerization, in-situ polymerization, etc. The thermosetting resin may include, for example, polyester resins, polyurethane resins, melamine resins, epoxy resins, diallyl phthalate resins, vinylester resins, and so forth. The resulting mixture may then be granulated and optionally coated with a hydrophilic macromolecular compound, such as alginic acid and salts thereof, carrageenan, pectin, gelatin and the like, semisynthetic macromolecular compounds such as methylcellulose, cationized starch, carboxymethylcellulose, carboxymethylated starch, vinyl polymers (e.g., polyvinyl alcohol), polyvinylpyrrolidone, polyacrylic acid, polyacrylamide, maleic acid copolymers, and so forth. The resulting thermochromic microcapsules typically have a size of from about 1 to about 50 micrometers, and in some aspects, from about 3 to about 15 micrometers. Various other microencapsulation techniques may also be described in U.S. Pat. No. 4,957,949 to Kamada, et al. and U.S. Pat. No. 5,431,697 to Kamata, et al., which are incorporated herein in their entirety by reference thereto for all purposes. Suitable microencapsulated thermochromic substances may also be obtained from Matsui Shikiso Chemical Co., Ltd. of Kyoto, Japan under the designation “Chromicolor.”

Thermochromic dyes are commercially available from various sources. In one aspect, for instance, thermochromic dyes marketed by Chromatic Technologies, Inc. of Ithaca, N.Y. may be incorporated into the cleansing composition.

The thermochromic dyes can be present on the absorbent article 20 in an amount sufficient to have a visual effect on the color of the composition. The amount or concentration of the dyes can also be increased or decreased depending upon the desired intensity of any color.

As described above, thermochromic dyes typically change from a specific color to clear at a certain temperature. If desired, other pigments or dyes can be added to the composition to provide a background color that remains constant independent of the temperature of the composition. By adding other pigments or dyes in combination with the thermochromic dyes to the composition, the thermochromic dyes can provide a color change at certain temperatures rather than just a loss of color should the thermochromic dye become clear. For instance, a non-thermochromic pigment, such as a yellow pigment, may be used in conjunction with a plurality of thermochromic dyes, such as a red dye and a blue dye. When all combined together, the composition may have a dark color. As the composition is increased in temperature, the red thermochromic dye may turn clear changing the color to a green shade (a combination of yellow and blue). As the temperature further increases, the blue thermochromic dye turns clear causing the cleansing composition to turn yellow.

It should be understood that all different sorts of thermochromic dyes and non-thermochromic pigments and dyes may be combined to produce a composition having a desired base color and one that undergoes desired color changes. The color changes, for instance, can be somewhat dramatic and fanciful. For instance, in one aspect, the composition can change from green to yellow to red, such as the colors of a stop light. Once the color of the composition turns red, a user would understand that a specific indication has been reached.

In an alternative aspect, however, the composition can include different thermochromic dyes all having the same color. As the temperature of the composition is increased, however, the shade or intensity of the color can change. For instance, the composition can change from a vibrant blue to a light blue to a clear color.

In addition to the above, it should be understood that many alterations and permutations are possible. Any of a variety of colors and shades can be mixed to undergo color changes as a function of temperature.

When thermochromic dyes are used in conjunction with non-thermochromic pigments or dyes, the non-thermochromic pigments or dyes may include any suitable pigments or dyes that do not interfere with the composition or with the function of the thermochromic dyes.

In another example, an electrical-based microclimate sensor can employ technologies known in the art for measuring temperature and displaying the measured temperature. Such a display can give the actual microclimate temperature or the display can indicate a temperature range. Additionally, an electrical-based microclimate sensor can be used to log or track the microclimate temperature the subject is experiencing over time.

In one aspect of the present invention, the microclimate sensor 120 translates the measured microclimate temperature into a recommendation ensuring that the subject has the proper amount of clothing or other coverings, such as that illustrated in FIG. 5. In one aspect, this recommendation may be made by simply replacing a temperature indication with a clothing recommendation. For example, if the microclimate temperature indication would otherwise indicate that the microclimate temperature is two degrees below normal temperature, the actual numerical microclimate temperature indication can be replaced by a short statement instructing the caregiver to add clothing. Additional indication schemes will be described below.

In another aspect of the present disclosure, the microclimate sensor 120 can be disposable to eliminate the need for keeping track of a durable sensor when not in use. The microclimate sensor 120 can be manufactured as a part of clothing or a disposable absorbent or other article.

In still another aspect of the present disclosure the microclimate sensor 120 can be a stand-alone unit that can be affixed to or placed adjacent to the skin or clothing of the subject by any suitable means. The microclimate sensor 120 can be sold or otherwise made available separately from clothing to allow the caregiver to use a microclimate sensor 120 where and when appropriate, or to allow the caregiver to place the microclimate sensor 120 somewhere other than, for example, the diaper area (e.g., the upper body).

Additionally, the clothing sufficiency indication system 10 can include a remote indicator 130 to be used with the microclimate sensor 120 to indicate to the caregiver whether the subject requires more or less clothing. In this aspect, the microclimate sensor 120 is adapted to be positioned within the subject's clothing and to transmit a microclimate temperature signal 126 to the remote indicator 130. The caregiver can then monitor the subject's microclimate remotely to determine whether the microclimate is normal without the need to remove the subject's clothing to observe the indication. The remote indicator 130 can be positioned on the exterior of the subject's clothing or in a more remote location, including as a separate device held by or accessible to the caregiver. The microclimate sensor 120 can transmit the microclimate temperature signal 126 to the remote indicator 130 either wirelessly, by wire, or by any other suitable manner. The remote indicator 130 can indicate temperature or clothing sufficiency in the manner described above with respect to FIGS. 4 and 5.

In any aspect of the present disclosure described herein, a sensor or the remote indicator 130 can include an alarm to indicate when a high setpoint, a low setpoint, or both setpoints are reached. The setpoints can be pre-set or set by a caregiver. The alarm can be audible, visual, tactile, or a wired or wireless transmission to another device such as a pager, a cell phone, an alarm clock, or another receiving unit of any suitable type.

The clothing sufficiency indication system 110 can also include a skin sensor 140 adapted to be disposed adjacent to the subject's skin. The skin sensor 140 includes a skin temperature sensing portion 144 that detects the subject's skin temperature. The skin sensor 140 then generates a skin temperature signal 146. In various aspects of the present disclosure, the skin temperature signal 146 can be directly readable by a caregiver, or the skin temperature signal 146 can be transmitted to another device to be described below.

The skin sensor 140 can also include an insulator 148 to separate the skin temperature sensing portion 144 from the subject's microclimate so that the subject's microclimate temperature does not affect the temperature reading of the skin sensor 140.

The skin sensor 140 can be printed on or otherwise made a part of clothing, including an absorbent article. In addition, the skin sensor 140 can be any suitable electrical, chemical, or other sensor.

For example, a chemical-based skin sensor 140 employing technology as described above can incorporate both temperature sensitive and temperature insensitive materials. In one example, a printed scale using temperature insensitive inks is overlaid by a temperature sensitive coating such as a thermochromic dye. As the sensor warms, the thermochromic dye begins to melt, which causes it to become clear, revealing a printed scale underneath.

In another example, an electrical-based skin sensor 140 can employ technologies known in the art such as a thermocouple for measuring temperature and displaying the measured temperature. Such a display can give the actual skin temperature or the display can indicate a temperature range. Additionally, an electrical-based skin sensor 140 can be used to log or track the skin temperature the subject is experiencing over time.

In one aspect of the present disclosure, the skin sensor 140 can be disposable to eliminate the need for keeping track of a durable sensor when not in use. The skin sensor 140 can be manufactured as a part of clothing or a disposable absorbent or other article.

Additionally, the remote indicator 130 to be used with the microclimate sensor 120 can also be used in conjunction with the skin sensor 140 to indicate to the caregiver whether the subject requires more or less clothing. In this aspect, the skin sensor 140 is adapted to be positioned adjacent to the subject's skin and to transmit a skin temperature signal 146 to the remote indicator 130. The caregiver can then monitor the subject's skin temperature remotely to determine whether the skin temperature is normal without the need to remove the subject's clothing to observe the indication. The remote indicator 130 can be positioned on the exterior of the subject's clothing or in a more remote location. The skin sensor 140 can transmit the skin temperature signal 146 to the remote indicator 130 or to the microclimate sensor 120 either wirelessly, by wire, or by any other suitable manner.

In another aspect of the present invention, the skin sensor 140 or the remote indicator 130 translates the measured skin temperature into the subject's core temperature to help ensure that the subject's core temperature is at an appropriate point. In one aspect, this translation can be made by simply replacing a skin temperature indication with a core temperature using standard skin-to-core temperature conversions. For example, if the skin temperature indication would otherwise indicate that the skin temperature is two degrees above normal temperature, the actual numerical skin temperature indication can be replaced by a core temperature. The remote indicator 130 can indicate temperature or clothing sufficiency in the manner described above with respect to FIGS. 4 and 5.

The clothing sufficiency indication system 110 can also include an ambient sensor 150 adapted to be disposed outside the clothing. The ambient sensor 150 includes an ambient temperature sensing portion 154 that detects the ambient temperature surrounding the subject. The ambient sensor 150 then generates an ambient temperature signal 156. In various aspects of the present disclosure, the ambient temperature signal 156 can be directly readable by a caregiver, or the ambient temperature signal 156 can be transmitted to another device to be described below.

The ambient sensor 150 can also include an insulator 158 to separate the ambient temperature sensing portion 154 from the subject's microclimate and skin so that the subject's microclimate or skin temperatures do not affect the temperature reading of the ambient temperature.

The ambient sensor 150 can be printed on or otherwise made a part of clothing, including an absorbent article. In addition, the ambient sensor 150 can be any suitable electrical, chemical, or other sensor.

For example, a chemical-based ambient sensor 150 employing technology as described above can incorporate both temperature sensitive and temperature insensitive materials. In one example, a printed scale using temperature insensitive inks is overlaid by a temperature sensitive coating such as a thermochromic dye. As the sensor warms, the thermochromic dye begins to melt, which causes it to become clear, revealing a printed scale underneath.

In another example, an electrical-based ambient sensor 150 can employ technologies known in the art for measuring temperature and displaying the measured temperature. Such a display can give the actual ambient temperature or the display can indicate a temperature range. Additionally, an electrical-based ambient sensor 150 can be used to log or track the ambient temperature the subject is experiencing over time.

In one aspect of the present disclosure, the ambient sensor 150 can be disposable to eliminate the need for keeping track of a durable sensor when not in use. The ambient sensor 150 can be manufactured as a part of clothing or a disposable absorbent or other article.

Additionally, the remote indicator 130 can also be used in conjunction with the ambient sensor 150 to indicate to the caregiver whether the subject requires more or less clothing. In this aspect, the ambient sensor 150 is adapted to be positioned adjacent to the subject and to transmit an ambient temperature signal 156 to the remote indicator 130. The caregiver can then monitor the subject's ambient temperature remotely to determine whether the ambient temperature is normal. The remote indicator 130 can be positioned on the exterior of the subject's clothing or in a more remote location. The ambient sensor 150 can transmit the ambient temperature signal 156 to the remote indicator 130 or to the microclimate sensor 120 either wirelessly, by wire, or by any other suitable manner. In another aspect of the present invention, the ambient sensor 150 can be a part of the remote indicator 130. The remote indicator 130 can indicate temperature or clothing sufficiency in the manner described above with respect to FIGS. 4 and 5.

In one aspect, the ambient sensor 150 and/or the remote indicator 130 can be connected to the microclimate sensor 120 through the subject's clothing using a pin or other mechanical device. The pin can be used to secure both the microclimate sensor 120 and the ambient sensor 150 and/or the remote indicator 130 to the clothing, and to transmit signals between the components.

The remote indicator 130 can be used to continuously monitor the subject's microclimate, skin, and/or ambient temperatures and to make recommendations with respect to the subject's clothing needs during the day and/or night. The remote indicator 130, or the microclimate sensor 120 itself, can also be adapted to recommend the type of clothing needed to ensure an optimal microclimate. Such a recommendation can be based on factors including the degree of variance from a normal temperature and the time of day. In addition, the recommendation can include a recommendation to seek medical assistance for microclimate or skin temperatures that are extremely high or low.

The remote indicator 130 of the clothing sufficiency indication system 110 can also be adapted to receive the microclimate, skin, and ambient temperature signals 126, 146, 156. The remote indicator 130 then can compare the microclimate and skin temperature signals 126, 146 to generate a skin-microclimate temperature gradient signal. The remote indicator 130 can also compare the microclimate and ambient temperature signals 126, 156 to generate an ambient-microclimate temperature gradient signal. The skin-microclimate temperature gradient signal and/or the ambient-microclimate temperature gradient signal can be used by the clothing sufficiency indication system 110 to determine the source of heating or cooling to help determine clothing sufficiency. The remote indicator 130 can indicate temperature or clothing sufficiency in the manner described above with respect to FIGS. 4 and 5.

More specifically, the clothing sufficiency indication system 110 can use the temperature signals 126,146,156 to determine the heat flux to or from the subject. This feature relates the subject's core and/or skin temperatures to the subject's microclimate and ambient temperatures to determine the heat flux between the subject's microclimate and the exterior of the subject's clothing. This determination establishes thermal gradients between the three interfaces (i.e., skin-microclimate, microclimate-clothing, clothing-ambient) and provides an accurate means of determining the thermal loss of the subject to the subject's environment (both microclimate and ambient).

This determination additionally can be used to provide temperature diagnostics for the subject. In this respect, this diagnostic tool can be used to identify high skin temperature relative to the microclimate and the ambient environment to indicate when a high skin temperature may be due to illness.

This information can be used to expand the idea of a thermal comfort measure by incorporating the thermal masses of the clothing/materials that line the body/environments.

These and other modifications and variations to the present disclosure may be practiced by those of ordinary skill in the art, without departing from the spirit and scope of the present disclosure, which is more particularly set forth in the appended claims. In addition, it should be understood that aspects of the various aspects of the present disclosure may be interchanged either in whole or in part. Furthermore, those of ordinary skill in the art will appreciate that the foregoing description is by way of example only, and is not intended to limit the disclosure so further described in such appended claims.

What is claimed:

1. A clothing sufficiency indication system for use in conjunction with a disposable absorbent article for a subject having skin, the system comprising:
a microclimate sensor adapted to be disposed on the disposable absorbent article, the microclimate sensor including a microclimate temperature sensing portion adapted to sense a microclimate temperature, wherein the clothing sufficiency indication system is adapted to generate a clothing sufficiency indication based on the microclimate temperature.

2. The indication system of claim 1, wherein the microclimate sensor includes thermochromic ink.

3. The indication system of claim 1, wherein the microclimate sensor is printed on a disposable absorbent article.

4. The indication system of claim 3, wherein the disposable absorbent article has an outer surface. and wherein the microclimate sensor is printed on the outer surface.

5. The indication system of claim 1, wherein the microclimate temperature sensor is adapted to generate a microclimate temperature signal.

6. The indication system of claim 5, further comprising a remote indicator adapted to receive at least the microclimate temperature signal and to indicate clothing sufficiency.

7. The indication system of claim 5, wherein the microclimate sensor includes a transmitter for transmitting the microclimate temperature signal.

8. The indication system of claim 7, wherein the transmitter is adapted to transmit the microclimate temperature signal wirelessly.

9. The indication system of claim 1, further comprising a skin sensor adapted to be disposed adjacent the skin, the skin sensor including a skin temperature sensing portion generating a skin temperature signal and a transmitter for transmitting the skin temperature signal.

10. The indication system of claim 9, further comprising a remote indicator adapted to receive the skin temperature signal and to indicate skin temperature.

11. The indication system of claim 9, further comprising a remote indicator adapted to receive the skin temperature signal and to indicate a core temperature of the subject.

12. The indication system of claim 9, further comprising a remote indicator for receiving the microclimate and skin temperature signals, wherein the remote indicator compares the microclimate and skin temperature signals to generate a skin-microclimate temperature gradient signal.

13. The indication system of claim 1, wherein the subject has clothing, and further comprising an ambient sensor adapted to be disposed outside the clothing, the ambient sensor including an ambient temperature sensing portion generating an ambient temperature signal and a transmitter for transmitting the ambient temperature signal.

14. The indication system of claim 13, further comprising a remote indicator for receiving the microclimate and ambient temperature signals, wherein the remote indicator compares the ambient and microclimate temperature signals to generate an ambient-microclimate temperature gradient signal.

15. The indication system of claim 1, wherein the clothing sufficiency indication system is adapted to indicate the microclimate temperature.

16. The indication system of claim 1, wherein the clothing sufficiency indication system is adapted to indicate a recommendation to seek medical assistance.

17. The indicator system of claim 1, wherein the clothing sufficiency indication includes a recommendation of a type of clothing to be added or removed.

18. A method for determining the clothing sufficiency of a subject, the method comprising:
providing a clothing sufficiency indication system including a microclimate sensor for measuring a microclimate temperature within clothing of the subject;
translating the microclimate temperature into a clothing sufficiency recommendation; and
communicating the clothing sufficiency recommendation to a caregiver.

19. The method of claim 18, wherein the microclimate sensor is printed on a disposable absorbent article.

20. The method of claim 18, wherein communicating the clothing sufficiency recommendation includes providing a remote indicator adapted to communicate with the microclimate sensor.

21. A method for determining the source of an elevated microclimate temperature of a microclimate within clothing, the method comprising:
measuring the microclimate temperature;
measuring a skin temperature of skin within the clothing;
determining a heat flux between the skin and the microclimate; and
informing a caregiver whether the elevated microclimate temperature is caused by an external source or a body source.

22. The method of claim 21, further comprising measuring an ambient temperature outside the clothing and determining a heat flux between the microclimate and an environment outside the clothing.

23. A method for determining the source of an elevated microclimate temperature of a microclimate within clothing, the method comprising:
measuring the microclimate temperature;
measuring an ambient temperature outside the clothing;
determining a heat flux between the microclimate and an environment outside the clothing; and
informing a caregiver whether the elevated microclimate temperature is caused by an external source or a body source.
